(19) United States
(12) Patent Application Publication
(10) Pub. No.: US 2012/0076877 A1
(43) Pub. Date: Mar. 29, 2012

(54) PHARMACEUTICAL COMPOSITIONS COMPRISING EXTRACTS OF SARCOPOTERIUM SPINOSUM, COMPONENTS THEREOF, AND USES THEREOF

(75) Inventors: Tovit Rosenzweig, Kedumim (IL); Zohar Kerem, Rehovot (IL); Dvir Taler, Kfar Haroe (IL)

(73) Assignee: Ariel-University Research and Development Company. Ltd., Ariel, IL (US)

(21) Appl. No.: 13/375,473

(22) PCT Filed: Jun. 8, 2010
(86) PCT No.: PCT/IB2010/052551

§ 371 Date: Nov. 30, 2011

Related U.S. Application Data
(60) Provisional application No. 61/185,605, filed on Jun. 10, 2009.
(60) Provisional application No. 61/253,521, filed on Oct. 21, 2009.

(51) Int. Cl. A61K 36/73; A61K 31/353; A61P 3/06; A61P 15/08; A61P 15/00; A61P 9/10; A61P 3/04; A61P 3/00; A61P 15/10; A61P 3/10; A61P 3/08

(52) U.S. Cl. 424/765; 514/456

(57) ABSTRACT

Disclosed are pharmaceutical compositions comprising extracts of Sarcopoterium spinosum, components of the extracts and uses of the pharmaceutical compositions as well as methods of making the compositions.

### RELATED APPLICATION

The present application gains priority from U.S. Provisional Patent Application Nos. 61/185,605 filed 10 Jun. 2009 and 61/253,521 filed 21 Oct. 2009, both which are included by reference as if fully set forth herein.

### FIELD AND BACKGROUND OF THE INVENTION

The present invention relates to the field of medicine, and more particularly to extracts of Sarcopoterium spinosum, pharmaceutical compositions comprising extracts of Sarcopoterium spinosum, components of the extracts and uses of the pharmaceutical compositions.

Diabetes mellitus (DM) is a common metabolic disease characterized by an absolute or relative reduction in the plasma insulin concentration. The decreased activity of insulin causes impairment in glucose metabolism, leading to hyperglycemia which is accompanied by impairment in protein and lipid metabolism. The main target tissues involved in glucose homeostasis are skeletal muscle, adipose tissue and the liver.

Insulin increases glucose uptake by hepatocytes, myotubes and adipocytes [12]. In muscle, the glucose is directed towards glycolysis and glycogenesis. This is achieved by regulation of several key enzymes in these pathways, such as glycogen synthase kinase 3β (GSK3β), an inhibitor of glycogen synthesis. Insulin blocks GSK3β activity by phosphorylating it on ser-9, leading to induction of glycogen synthesis [17, 18].

In adipocytes, insulin directs glucose towards glycolysis and lipogenesis pathways, while inhibiting lipolysis, a catabolic process that increases free fatty acid release to the plasma [9]. Insulin also inhibits other catabolic pathways, including hepatic and muscular glycogenolysis, thus eliminating glucose flux to the plasma [22]. The decreased insulin activity causes impairment in glucose metabolism, leading to severe complications such as atherosclerosis, nephropathy retinopathy and neuropathy.

Diabetes is regarded as a major cause of premature morbidity and mortality in developed countries, affecting more than 170 million individuals worldwide. It is estimated that in 2030, more than 330 million patients will be diagnosed as diabetics. The most common form of diabetes is type 2 diabetes mellitus, accounting for more than 90% of diabetes cases. The disease causes both medical and socioeconomical burdens brought about by the common complications of diabetes such as atherosclerosis, nephropathy and neuropathy. As a result of these complications, diabetes is considered to be one of the major causes for premature illness and mortality.

Today, several drugs are available for the treatment of diabetes, including metformin, rosiglitazone, GLP-1 analogs and insulin. The conventional medical approaches available today deals with the clinical manifestations of diabetes rather than a cure of the disease. Thus, many herbal medicines have found their way into the world market as alternatives to prescribed drugs that are currently available for treating various disorders [1]. Accordingly, their use in western countries and the costs incurred have increased each year.

While there is evidence that some herbal medicines have physiological effects [4, 6], the efficacy of most of these folk medicine plants in treating diseases such as diabetes has only rarely been scientifically tested and validated [2]. Consequently, knowledge of the efficacy, specific effects obtained by using the herbs, and their mechanisms of action is very limited, and is based on information collected from local medicinal plant practitioners [3,4].

Because of the ever-increasing number of diabetes patients, it is of major medical and economic importance to find new strategies for the treatment and even prevention of the disease.

During the last 20 years, several extensive ethno-botanical surveys have been carried out in Jordan and Israel in order to document and identify the local medicinal plant species used by traditional Arab medicine, their properties and usage [5,6]. Sarcopoterium spinosum (L.) sp. has been mentioned in all of these ethnobotanical surveys as a medicinal plant, used by traditional Arab and Bedouin medicine for the treatment of diabetes, problems in the digestive system, pain relief or cancer [3,4].

Sarcopoterium spinosum (L.) sp., also known as thorny burnet (syn: Poterium spinosum L.) [3,5], is an abundant and characteristic species of the semi-steppe shrublands (phrygana) and Batha of the Eastern Mediterranean region. S. spinosum is a chamaephyte of the Rosaceae family. Its branches are wooden, end in branched thorns and grow to a length of 30-40 cm. In the summer the green winter leaves at the end of the branches develop into thorns and are replaced by tiny leaves [6].

Despite the well-documented usage of S. spinosum root extract for treating diabetes in Arab folk medicine [7], very few studies have confirmed this information and measured the antidiabetic activity of S. spinosum extract using scientific tools.

### SUMMARY OF THE INVENTION

According to an aspect of some embodiments of the invention there is provided a method of increasing insulin secretion in a subject in need thereof, the method comprising administering a pharmaceutically-effective amount of a composition comprising an extract of S. spinosum to the subject.

According to an aspect of some embodiments of the invention, there is provided a method of decreasing blood insulin in a subject in need thereof, the method comprising administering a pharmaceutically-effective amount of an extract of S. spinosum to the subject.

According to an aspect of some embodiments of the invention, there is provided a method of increasing pancreatic cell proliferation in a subject in need thereof, the method comprising administering a pharmaceutically-effective amount of an extract of S. spinosum to the subject.

According to an aspect of some embodiments of the invention, there is provided a method of inhibiting lipolysis in an adipocyte in a subject in need thereof, the method comprising administering a pharmaceutically-effective amount of an extract of S. spinosum to the subject.

According to an aspect of some embodiments of the invention, there is provided a method of inducing glucose uptake in a cell of a subject in need thereof, the method comprising administering a pharmaceutically-effective amount of an extract of S. spinosum to the subject. In some embodiments, the cell is an adipocyte. In some embodiments, the cell is a hepatocyte. In some embodiments, the cell is a myotube.

According to an aspect of some embodiments of the invention, there is provided a method of increasing glycogen synthesis in a subject in need thereof, the method comprising administering a pharmaceutically-effective amount of an extract of S. spinosum to the subject.

According to an aspect of some embodiments of the invention, there is provided a method of reducing plasma glucose levels in a subject in need thereof, the method comprising administering a pharmaceutically-effective amount of an extract of S. spinosum to the subject.

According to an aspect of some embodiments of the invention, there is provided a method of preventing (or delaying the onset of) diabetes (in some embodiments type 1 diabetes, in some embodiments type 2 diabetes) in a subject, the method comprising administering a pharmaceutically-effective amount of an extract of S. spinosum to the subject.

According to an aspect of some embodiments of the invention, there is provided a method of increasing the fertility of a diabetic female subject in need thereof, the method comprising administering a pharmaceutically-effective amount of an extract of S. spinosum to the subject.

According to an aspect of some embodiments of the invention, there is provided a method of raising levels of AMPK (AMP-activated protein kinase) in a subject, the method comprising administering a pharmaceutically-effective amount of an extract of S. spinosum.

According to an aspect of some embodiments of the invention, there is provided a method of improving glucose tolerance in a subject in need thereof, the method comprising administering a pharmaceutically-effective amount of an extract of S. spinosum to the subject.

According to an aspect of some embodiments of the invention, there is provided a method of preventing (or delaying the onset of) atherosclerosis in a subject, the method comprising administering a pharmaceutically-effective amount of an extract of S. spinosum to the subject.

According to an aspect of some embodiments of the invention, there is provided a method of decreasing weight gain in a subject in need thereof, the method comprising administering a pharmaceutically-effective amount of an extract of S. spinosum to the subject.

According to an aspect of some embodiments of the invention, there is provided a method of decreasing food consumption in a subject in need thereof, the method comprising administering a pharmaceutically-effective amount of an extract of S. spinosum to the subject.

According to an aspect of some embodiments of the invention, there is provided a method of increasing longevity in a subject, the method comprising administering a pharmaceutically-effective amount of an extract of S. spinosum to the subject.

According to an aspect of some embodiments of the invention, there is provided a method of improving blood chemistry of a subject in need thereof, the method comprising administering a pharmaceutically-effective amount of an extract of S. spinosum to the subject. In some embodiments, improving blood chemistry includes reducing free fatty acids in the blood of the subject.

According to an aspect of some embodiments of the invention, there is provided a method of increasing the libido of a female subject, the method comprising administering a pharmaceutically-effective amount of an extract of S. spinosum to the subject. In some embodiments, the subject suffers from hyperlipidemia.

According to an aspect of some embodiments of the invention, there is provided a method of treating erectile dysfunction of a male subject, the method comprising administering a pharmaceutically-effective amount of an extract of S. spinosum to the subject. In some embodiments, the subject suffers from hyperlipidemia.

According to an aspect of some embodiments of the invention, there is provided a method of reducing or preventing obesity in a subject in need thereof, the method comprising administering a pharmaceutically-effective amount of an extract of S. spinosum to the subject.

In some embodiments, the subject of any of the methods described herein is a human. In some embodiments, the subject of any of the methods described herein is a non-human animal.

In some embodiments, the subject of any of the methods described herein is susceptible to development of diabetes (in some embodiments type 1 diabetes and in some embodiments type 2 diabetes). In some embodiments, the subject is diabetic. In some embodiments, the subject is not diabetic.

In some embodiments, the subject of any of the methods described herein is susceptible to developing hyperlipidemia. In some embodiments, the subject has hyperlipidemia.

According to further features in the described preferred embodiments, use or administering of an extract of S. spinosum to a subject comprises administering the extract by a route selected from the group consisting of the oral, transdermal, intravenous, subcutaneous, intramuscular, intranasal, intraauricular, sublingual, rectal, transmucosal, intestinal, intraauricular, buccal, intramedullar, intrathecal, direct intraventricular, intraperitoneal, and intraocular routes. Preferably, administering is effected by the oral, transdermal, buccal, transmucosal, rectal or sublingual routes. More preferably, administering is effected by the oral route.

According to some embodiments, there are provided methods for preparing pharmaceutical compositions comprising an extract of S. spinosum , for example, useful for implementing any of the methods described above. In some embodiments, the method of preparing the pharmaceutical composition comprises mixing an extract of S. spinosum with a pharmaceutically-acceptable carrier, and optionally one or more suitable excipients. Preferably, the carrier is a liquid carrier. In some embodiments, an extract of S. spinosum is a composition of S. spinosum , for example, a hot water extract (tea) is both an extract and a composition where the pharmaceutically-acceptable carrier is the water used in the extraction.

According to an aspect of some embodiments of the invention, there is provided an extract of S. spinosum for use as a medicament for the treatment of a condition, for example useful for implementing any of the methods described above.

According to an aspect of some embodiments of the invention, there is provided a pharmaceutical composition comprising an extract of S. spinosum for treatment of a condition, for example useful for implementing any of the methods described above. The composition may comprise, in addition to the extract of S. spinosum , a pharmaceutically-acceptable carrier, and optionally one or more suitable excipients. Preferably, the carrier is a liquid carrier.

According to an aspect of some embodiments of the invention, there is provided for the use of an extract of S. spinosum in the manufacture of a pharmaceutical composition for treatment of a condition, for example useful for implementing any of the methods described above. The pharmaceutical composition may comprise, in addition to the extract of S. spinosum, a pharmaceutically-acceptable carrier, and optionally one or more suitable excipients. Preferably, the carrier is a liquid carrier.

In some embodiments, the condition is selected from the group consisting of conditions susceptible to: increasing insulin secretion, decreasing blood insulin, increasing pancreatic cell proliferation, inhibiting lipolysis in an adipocyte, inducing glucose uptake in a cell, increasing glycogen synthesis, reducing plasma glucose levels, preventing or delaying the onset of diabetes, raising levels of AMP-activated protein kinase, improving glucose tolerance, preventing or delaying the onset of atherosclerosis, decreasing weight gain, decreasing food consumption, increasing longevity, improving blood chemistry, reducing free fatty acids in the blood, reducing obesity and preventing obesity.

In some embodiments, the condition is selected from the group consisting of insufficient insulin secretion, decreased blood insulin, insufficient pancreatic cell proliferation, insufficient glucose uptake in cells, insufficient glycogen synthesis, high plasma glucose levels, diabetes, decreased fertility of a diabetic female, reduced AMP-activated protein kinase, insufficient glucose tolerance, athersclerosis, weight gain, obesity, excessive food consumption, poor blood chemistry, excessive free fatty acids in the blood, decreased female libido, male erectile dysfunction and hyperlipidemia.

Additional inventions are described hereinbelow.

As used herein, the term “extract of S. spinosum” and equivalent terms refers to an extract, or to a component of such extract (however provided, e.g., isolated from a natural extract of S. spinosum , isolated from another source, or synthesized) or a combination of two or more such components, as long as the “extract of S. spinosum” is effective for the prescribed use.

Any suitable pharmaceutically-active extract of S. spinosum may be used in implementing the teachings of the invention.

In some embodiments, an extract of S. spinosum is an extract of a part or parts of S. spinosum, for example, flowers, leaves, stems, branches, fruit and roots. In some embodiments, a preferred extract is an extract of a root of S. spinosum.

In some embodiments, an extract of S. spinosum is an extract of a part or parts of S. spinosum, acquired for example, by distillation (e.g., steam distillation, vacuum distillation), by extraction (solvent extraction, alcohol extraction, oil extraction, super critical extraction, water extraction and hot water extraction). In some embodiments, a preferred such extract is the hot water extract (tea) of a part of S. spinosum.

In some embodiments, a preferred extract is the hot water extract (tea) of the root of S. spinosum.

In some embodiments, an extract of S. spinosum is one or more pharmaceutically-active components of an extract of a part or parts of S. spinosum as described above. In some embodiments, one or more of the pharmaceutically-active components are natural products that have been separated, isolated and or purified, for example using methods known in the art, for example purification, sedimentation or extraction.

In some embodiments, one or more of the pharmaceutically-active components are synthetic compounds synthesized to have substantially the same activity as such a natural product, generally being identical or substantially identical to such a natural product.

In some embodiments, at least one pharmaceutically-active component is a guanidine. In some embodiments, at least one pharmaceutically-active component is or is a derivative of isoamylene guanidine (galegine) as isolated from Galega officinalis.

In some embodiments, at least one pharmaceutically-active component is a catechin or derivative thereof In some embodiments, at least one pharmaceutically-active component is or is a derivative of catechin or epicatechin.

In some embodiments, an extract of S. spinosum is coadministered or is provided in a dosage form together with at least one additional (non-extract of S. spinosum) active pharmaceutical ingredient (API). In some embodiments, an additional API is selected from the group consisting of rosiglitazone, pioglitazone, a sulfonyl urea such as glipizide or glibenclamide, a dipeptidyl peptidase-4 inhibitor such as sitagliptin or a meglitinide such as repaglinide.

Unless otherwise defined, all technical and scientific terms used herein have the same meaning as commonly understood by one of ordinary skill in the art to which the invention pertains. In case of conflict, the specification, including definitions, will control. The terms “pharmacologically effective” and “pharmaceutically effective” are herein used interchangeably. The terms “pharmacologically active” and “pharmaceutically active” are herein used interchangeably. Herein, the terms “composition” and “pharmaceutical composition” are generally used interchangeably.

Some embodiments of the invention comprise administering a pharmaceutically-effective amount of an extract of S. spinosum in order to achieve a beneficial effect. In some embodiments, a beneficial effect includes for example, in some embodiments treating a condition, curing a condition, preventing a condition, treating symptoms of a condition, curing symptoms of a condition, ameliorating symptoms of a condition, treating effects of a condition, ameliorating effects of a condition, and preventing results of a condition.

### BRIEF DESCRIPTION OF THE FIGURES

Some embodiments of the invention are herein described, by way of example only, with reference to the accompanying figures. The description, together with the figures, makes apparent how embodiments of the invention may be practiced to a person having ordinary skill in the art. The figures are for the purpose of illustrative discussion of embodiments of the invention and no attempt is made to show structural details of an embodiment in more detail than is necessary for a fundamental understanding of the invention.

In the Figures:

FIG. 1A is a line graph showing cell viability as a percentage optical density (OD) on days 3 and 5, as compared to that on seeding;

FIG. 1B is a bar chart showing the effect of various concentrations S. spinosum extract on cell viability as percentage of control at days 3 and 5;

FIG. 2A is a bar chart showing the effect of various concentrations of S. spinosum extract on basal level insulin secretion;

FIG. 2B is a bar chart showing the effect of various concentrations of S. spinosum extract on glucose/forskolin insulin secretion;

FIG. 2C is a bar chart showing the effect of various concentrations of S. spinosum extract on preproinsulin mRNA expression;

FIG. 3A is a Western blot showing the effect of S. spinosum extract on GSK3β phosphorylation using anti-pGSK (ser 9) or anti-actin;

FIG. 3B is a bar chart showing the effect of insulin and S. spinosum extract on GSK3β phosphorylation;

FIG. 4A is a bar chart showing the effect of insulin and S. spinosum extract on free fatty acid release in the presence and absence of isoproterenol;

FIG. 4B is a line graph showing the effect of various concentrations of S. spinosum extract on free fatty acid release in the presence and absence of isoproterenol;

FIG. 5A is a bar chart showing the effect of insulin and S. spinosum extract on glucose uptake in hepatocytes;

FIG. 5B is a bar chart showing the effect of insulin and S. spinosum extract on glucose uptake in myoblasts;

FIG. 5C is a bar chart showing the effect of insulin and S. spinosum extract on glucose uptake in adipocytes;

FIG. 6 is a graph showing the effect of a composition comprising S. spinosum extract on the intraperitoneal glucose tolerance test (IPGTT);

FIG. 7 is a bar chart showing the effect of a composition comprising S. spinosum extract on weight gain;

FIG. 8 is a bar chart showing the effect of a composition comprising S. spinosum extract on food consumption;

FIG. 9 is a bar chart showing the effect of a composition comprising S. spinosum extract on free fatty acid blood concentration in vivo;

FIG. 10 is a bar chart showing the effect of a composition comprising S. spinosum extract on blood insulin; and

FIG. 11 is a graph showing the effect of short-term administration of a composition comprising S. spinosum extract on diabetic mice.

### DESCRIPTION OF SOME EMBODIMENTS OF THE INVENTION

The present invention relates to extracts of Sarcopoterium spinosum, compositions comprising such extracts and uses thereof.

Several studies carried out in the 1960's and 1980's proved that an extract of S. spinosum roots exhibits a hypoglycemic effect in rats [1, 11-13]. These studies on S. spinosum and diabetes were not completed, and much information on the specific effects and mechanisms of action is lacking Reher et al. [14,15] isolated and identified 3 known triterpenoids from the root as the active hypoglycemic substances. These studies indicate that S. spinosum is an antidiabetic agent. However, there are no indications of the mechanism of action of the extract, its targets in cells, or whether it can improve glycemic control of type 1, type 2 or both types of diabetes.

It was not previously known whether triterpenoid compounds, as isolated by Reher et al., [14, 15] alone or in combination with other unidentified yet compounds, mediate the various metabolic effects of S. spinosum extract. The specific pathway affected by these compounds and their molecular mechanism of action have not been previously studied or clarified.

The present inventors studied the effect of compositions comprising S. spinosum extracts on various physiological functions, including insulin secretion, pancreatic β-cell viability, GSK3β phosphorylation, lipolysis and glucose uptake, by treating RINm pancreatic β-cells, L6 myotubes, 3T3-L1 adipocytes and AML-12 hepatocytes with different doses of S. spinosum extract, as described in detail in the Examples section below.

In vitro, S. spinosum compositions were found to have insulin-like effects in skeletal muscle, adipose tissue and hepatocytes, which play important roles in the maintenance of glucose homeostasis. The extract increased glucose uptake in hepatocytes, myotubes and differentiated adipocytes. The S. spinosum compositions increased GSK3β phosphorylation in myotubes, indicating glycogen synthesis. Furthermore, the compositions inhibited lipolysis in adipocytes, as occurs in response to insulin. In addition, the extract increased pancreatic β cells viability and insulin secretion.

The RINm insulinoma cell line was used as a model for studying the effect of the S. spinosum compositions on β-cell function. The effects of the S. spinosum compositions on pancreatic β-cell proliferation and insulin secretion were measured.

S. spinosum compositions were found to increase insulin secretion in pancreatic beta-cells, and has insulin-like effects in classic insulin-responsive tissues. The present results show that S. spinosum compositions increased basal as well as glucose/forskolin-induced insulin secretion, and increased cell viability. The finding showing increased β-cell proliferation by the S. spinosum compositions is important, since diabetes mellitus is characterized by a loss of β-cell viability [24, 25].

One of the major physiological responses of cells to insulin induction is an increase in glucose uptake. The present inventors have demonstrated for the first time that S. spinosum compositions have insulin-like effects in skeletal muscle, adipose tissue and hepatocytes, which are classic target tissues of insulin and play important roles in the maintenance of glucose homeostasis. The effect of S. spinosum compositions on glucose uptake was measured in hepatocytes, myoblasts and adipocytes, and compared to the effect of insulin. The compositions were found to increase glucose uptake in each cell type.

Phosphorylation of glycogen-synthase kinase 3-β (GSK3β) on the serine residue (ser-9) was measured in order to monitor glycogen synthesis. S. spinosum compositions were found to increase GSK3β phosphorylation in L6 myotubes. Furthermore, compositions inhibited isoproterenol-induced lipolysis in adipocytes, as occurs in response to insulin which is a lipogenic, anti-lipolytic hormone.

The basal lipolysis rate in adipocytes is very low, and can barely be measured. The present inventors therefore induced lipolysis using the adrenergic agonist isoproterenol in order to analyze the compositions' effect on lipolysis [20, 21], and measured the effect of insulin as a positive control and of S. spinosum compositions on isoproterenol-induced lipolysis. S. spinosum compositions were found to inhibit isoproterenol-induced lipolysis in 3T3-L1 adipocytes, and induced glucose uptake in these cells as well as in AML-12 hepatocytes and L6 myotubes.

In vivo studies show improved glucose tolerance in A^{y} mice which were chronically administered a composition comprising S. spinosum extract.

Additional in vivo studies in mice investigated the effect of administration of a composition comprising a S. spinosum extract on weight gain; food intake; free fatty acid blood concentration; and blood insulin.

Compositions comprising S. spinosum extract were found to decrease weight gain, decrease food consumption, reduce fasting fatty acid blood concentration, reduce fasting blood insulin concentration, and to generally improve the appearance and metabolic profiles of the mice. The results suggest that in some embodiments such compositions have various beneficial effects such as increasing insulin secretion, decreasing blood insulin, increasing pancreatic cell proliferation, inhibiting lipolysis in an adipocyte, inducing glucose uptake in a cell, increasing glycogen synthesis, reducing plasma glucose levels, preventing (or delaying the onset of) diabetes, preventing (or delaying the onset of) atherosclerosis, decreasing weight gain, decreasing food consumption, increasing longevity, improving blood chemistry and/or reducing or preventing obesity.

The present inventors further aim to identify and isolate active compounds which mediate the anti-diabetic effects of Sarcopoterium spinosum extract; to analyze the specific anti-diabetic activity of each identified active compound; and to clarify molecular targets of the active compounds and their mechanisms of action.

It is hypothesized that the various beneficial effects of the extracts and compositions comprising the extracts are evoked by one or more different active compounds and that, in some cases, two or more different compounds may act in concert. Triterpenoids, of which some have been identified and reported in S. spinosum by Reher et al. [14], make promising candidates. There are several reports showing that certain naturally occurring triterpenoids, which are found in medicinal plants such as Panax ginseng [26], Platycodi radix [27] and Radix astragali [28] act as antidiabetic agents. In the extracts, the Inventors have identified catechins, epicatechins and derivatives thereof that may be responsible for at least some of the beneficial effects. The Inventors hypothesize that the extracts may comprise guanidines such as galegine or derivatives thereof. Other yet unidentified compounds may mediate the various beneficial effects.

To identify the active compounds, a hot water extract of the root of S. Spinosum is prepared and separated into 3 fractions using HPLC. Pharmaceutical activity of each fraction is assayed by measuring its effects on glucose uptake, lipolysis and insulin secretion in-vitro. The active fractions are separated further for complete analysis of the active compounds. The pharmaceutical functions of the identified compounds are evaluated in-vivo using mice model of diabetes. Molecular mechanisms of action are investigated by following changes in mRNA and protein expression and phosphorylation profile, by PCR and protein array platforms induced by the active compounds.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### EXAMPLES

Reference is now made to the following examples, which together with the above descriptions illustrate some embodiments of the invention in a non limiting fashion. Experiments were performed using standards methods and devices with which a person having ordinary skill in the art is familiar.

### Methods and Materials

### Chemicals, Kits and Reagents

Isoproterenol and inhibitors of proteases and phosphatases were purchased from Sigma (Sigma-Aldrich, St. Louis Mo., USA). An ELISA kit for insulin measurement was purchased from Mercodia (Uppsala, Sweden), and for leptin measurement, from ASSAYPRO LLC (St. Charles, Mo., USA). NEFA-C kit for free fatty acid determination was obtained from Wako Chemicals USA, Inc. (Richmond Va., USA), phospho-GSK3β (ser-9) antibody was obtained from Santa-Cruz Biotechnology (Santa Cruz, Calif., USA). Cell extraction buffer and a phospho-GSK3β (ser-9) ELISA detection kit were purchased from Calbiochem (a subsidiary of Merck KGaA, Darmstadt, Germany). A cell proliferation kit was obtained from Biological Industries (Beit Haemek, Israel). Forskolin, (palmitic acid) and anti-actin were purchased from MP Biomedicals (Irvine, Calif., USA). Reagents and media for cell cultures were obtained from Biological Industries (Beit Haemek, Israel).

### Plant Material

In order to obtain the roots, Sarcopoterium spinosum (L.) sp. plants were uprooted from the open area outside the Ariel University Center in Samaria, Israel. The plants were identified by the botanical staff of the University Center as Sarcopoterium spinosum (L.) sp. Taxonomic identity of the plant was made by comparison with identified voucher specimen (No. 313158-313177) from the Herbarium of Middle Eastern Flora (Israel National Herbarium) at the Hebrew University of Jerusalem, Jerusalem, Israel.

### Extract and Composition Preparation

In addition to the data published in ethnobotanical surveys [3, 5-7, 9], three Bedouin medicinal plant healer from the Samaria and Negev regions in Israel were interviewed regarding methods of extraction. The plants were shown to the healers, and their identity was confirmed. The plants were collected and an extract prepared according to the instructions of the healers. Specifically, the largest plants were chosen during the green season. 100 g fresh S. spinosum roots were cut into small pieces on the same day and boiled in 1 L of water for 30 minutes. The solutions were left for 3 h and the red supernatants were transferred through cloth to a sterile bottle without disturbing the pellet, and kept at 4° C. The resulting hot water extracts (tea) of S. spinosum are also considered to be a composition as described herein.

The S. spinosum extract prepared as above was diluted to 0.001-10% (V/V) to prepare compositions comprising an extract of S. spinosum for further study.

100 ml of an S. spinosum extract as described above was lyophilized in the usual way, yielding 3.975 g of a powdery root extract of S. spinosum.

### Cell Culture

3T3-L1 pre-adipocytes were cultured and differentiated as described in references 8 as well as 16, 19, 20 and 21. Briefly, cells were grown to confluence in Dulbecco's Modified Eagle's Medium (DMEM) containing 10% fetal calf serum (FCS), 2 mM glutamine and 1% ampicillin. Two days after full confluence, the cells were induced to differentiate by 3 days incubation in DMEM containing 10% FCS, 0.5 mM isobutylmethylxanthine (IBMX), 1 μM dexamethasone and 100 nM insulin. This was followed by 2 days of incubation in DMEM containing 10% FCS and 100 nM insulin. The cells were grown for an additional 5-9 days in DMEM containing 10% FCS. 3T3-L1 adipocytes were used for the experiment 10-14 days after the initiation of differentiation, when 80-90% of cells exhibited adipocyte morphology.

L6 myoblasts were grown in MEM-α containing 25 mM glucose, 10% FCS, 2 mM glutamine and 1% ampicillin. Experiments were performed on differentiated myotubes. In order to induce differentiation, myoblasts were incubated in MEM-α containing 25 mM glucose and 2% FCS for 4 days, followed by an additional 24 h incubation in a MEM-α starvation medium containing 5 mM glucose and 2% FCS. AML-12, a nontransformed hepatocyte cell line were maintained in DMEM containing 25 mM glucose, 10% FCS, 2 mM glutamine and 1% ampicillin.

Rat insulinoma (RINm) cells were grown in RPMI medium containing 10% FCS, 11 mM glucose, 2 mM HEPES, 1 mM sodium pyruvate, 2 mM glutamine and 1% ampicillin. All cells were grown at 37° C. in a humidified atmosphere containing 5% CO₂.

### Free Fatty Acid Release Assay

Differentiated adipocytes were preincubated for 12 hours in DMEM containing 2% fatty acid-free bovine serum albumin, in the absence of FCS. Lipolysis was stimulated by treating cells with 10 μM isoproterenol for 1 hour, in the absence or presence of insulin (100 nM) or a composition (S. spinosum extract diluted to 0.001-1% V/V). Free fatty acids (FFA) released into the culture media were measured immediately using a NEFA-C kit, according to the kit instructions.

### Western Immunoblot Analysis

Differentiated L6 myotubes were treated with 10 μg/ml insulin or a composition (S. spinosum extract diluted to 0.1, 0.01% V/V) for the indicated times. Protein lysates were prepared using cell extraction buffer supplemented with protease and phosphatase inhibitors. The samples were homogenized and centrifuged at 14,000 rpm for 20 min. The supernatant was collected and protein concentration was measured using the Bradford method. 20 μg protein per lane was separated by SDS-polyacrylamide gel electrophoresis. Proteins were electrophoretically transferred onto nitrocellulose membranes. The membranes were blocked in 10% dry milk, incubated with the appropriate antibodies and immunodetected using the enhanced chemiluminescence method.

### Detection of GSK3β (Serine-3) Phosphorylation

Differentiated L6 myotubes were treated with 100 nM insulin or composition (S. spinosum extract diluted to 0.1% V/V) for 20 min. Protein lysates were prepared using cell extraction buffer. Phosphorylation of GSK3β (ser-3) was determined using an ELISA detection kit, according to the manufacturer's instructions.

### Insulin Secretion Studies

RINm cells (10⁵ cells/ml) were cultured in 24-well plates for 48 h before measurement of insulin secretion. The cells were treated with composition (S. spinosum extract diluted to 0.001-1% V/V) for 1 or 24 h, followed by preincubation in Krebs-Ringer bicarbonate Hepes (KRBH) buffer containing 25 mM NaHCO₃, 115 mM NaCl, 4.7 mM KCl, 2.56 mM CaCl₂, 1.2 mM MgSO₄, 20 mM HEPES, 0.1% BSA and 2.8 mM glucose for 30 min. The supernatant was collected. The cells were then incubated in KRBH buffer containing 15 mM glucose and 10 μM forskolin for an additional 30 min. The supernatant was collected and the insulin concentration in the basal state and after induction was measured using an insulin immunoassay kit.

### Cell Proliferation Assay

Cell proliferation was assessed by a cell proliferation kit which is based on the ability of viable cells to reduce tetrazolium salt (XTT) into colored compounds of formazans. The dye intensity is measured by an ELISA reader. RINm cells were subcultured on a 96-well plate at a concentration of 5×10⁴ cells/ml in growth medium containing different concentrations of S. spinosum extract (0.001-1% V/V). The medium was discarded and replaced each day with medium containing fresh S. spinosum extract. After 3 or 5 days of incubation, 100 μl reaction solution (which contains XTT reagent and activation solution) was added to each well. After 4 h of incubation, the optical density (OD) of the samples was measured using a microplate reader (Tecan Group Ltd., Männedorf, Switzerland) at a test wavelength of 492 nm and a reference wavelength of 690 nm.

### Glucose Uptake

Differentiated adipocytes, L6 myoblasts and AML12 cells were each preincubated for 6 hours in low glucose (4.5 mM), serum-free DMEM containing 1% BSA, and then treated with either 100 nM insulin or compositions comprising different concentrations of S. spinosum extract (0.001-1% V/V).

Glucose uptake was measured in triplicate samples in six-well plates using [³H]2-deoxy-D-glucose (1mCi/ml; American Radiolabeled Chemicals, St. Louis, Mo., USA). After insulin or S. spinosum extract treatment, the cells were washed three times with warm (37° C.) PBS, the final wash being replaced immediately with 0.75 ml PBS containing 0.5 μCi/ml [³H]2-deoxy-D-glucose and glucose at a concentration of 0.1 mM. The cells were then incubated for 10 min at 37° C., washed three times with cold (4-6° C.) PBS, and then lysed by addition of 1 ml of 0.1% SDS and incubated for 30 min in 37° C. The contents of each well were transferred to counting vials, and 3.5 ml scintillation fluid was added to each vial and vortexed. Samples were counted in the ³H window of a Tricarb scintillation counter. Values were normalized to the protein content of each well.

Analysis of mRNA Expression by PCT Reactions

Total RNA was extracted from RINm cells using PerfectPure tissue RNA Kit (5PRIME, Gaithersburg, Md., USA). 2.5 ng of total RNA were reverse transcripted by oligo dT priming (Stratascript 5.0 multi-temperature reverse transcriptase, Stratagene) according to the manufacturer's instructions. Real-time PCR amplification reactions were performed using SYBRGreen Master mix (Rovalab GmbH, Teltow, Germany) by the MxPro QPCR instrument (Stratagene, an Agilent Technologies Division, Cedar Creek, Tex., USA).

Primer sequences and their respective PCR fragment length were as follows: Preproinsulin (160 bp): forward 5′-tcaaacagcacctt-3′, reverse 5′-agtgccaaggtctga-3′. Rat HPRT was used as housekeeping gene (130 bp): forward 5′-aggccagacttgttggat-3, reverse 5′-gcttttccactttcgctgat-3′.

### Animal Experiments

The Animal House at the Ariel University Center operates in compliance with the rules and guidelines set down by the Israel Council for Research in Animals (Israel Ministry of Health), based on the US National Institutes of Health's Guide for the Care and Use of Laboratory Animals, DHEW (NIH, Pub. 78-23). All studies were approved by the institute committee on use and care of animals, institutional license number: IL 090908.

KK-A^{y} strain mice were purchased from the Jackson Laboratory (Bar Harbor, Me., USA) at age of 4 weeks. The mice were housed in an animal laboratory with a controlled environment of 20-24° C., 45-65% humidity, and a 12 h (07:30-19:30) light/dark cycle. Unless otherwise stated, all experiments were performed on males, which were housed individually. Mice were separated into two groups (control and test; 8-10/group). The mice were fed ad libitum rodent chow, and were given ad libitum drinking water in the control group or S. spinosum composition (the extract described above, made by boiling 100 gram fresh S. spinosum root in 1 liter water) instead of their drinking water in the test group. Average consumption of water or the composition was measured, and found to be 15 ml/day, which in the case of the composition is equivalent to 600 mg/kg/day powdery lyophilized S. spinosum extract. At age 13 and 17 weeks, intraperitoneal glucose tolerance test (IPGTT) was performed. Food consumption from 5 consecutive days was used to calculate average daily food intake. At age 17 weeks, blood was collected from the orbital plexus. Serum was then prepared and stored at 80° C. until assayed for insulin, leptin and free fatty acids.

### Chromatography and Mass Spectroscopy

LC/MS experiments were carried out on a Thermo Electron LTQ-Orbitrap Discovery hybrid FT mass spectrometer (San Jose, Calif., USA) equipped with an Accela High Speed LC system (Thermo Fisher Scientific Inc., Waltham Mass., USA). The mass spectrometer was equipped with an electrospray ionization ion source, and operated in the negative ionization mode. Ion source parameters: spray voltage 3.5 kV, capillary temperature 250° C., source fragmentation was 35V, sheath gas rate (arb) 30, and auxiliary gas rate (arb) 10. Mass spectra were acquired in the m/z 150-2000 Da range. The LC-MS system was controlled and data was analyzed using Xcalibur software (Thermo Fisher Scientific Inc., Waltham Mass., USA).

Accela LC coupled to the MS served for chromatographic separation, consisting of an Accela Pump, Accela Autosampler and Accela PDA detector. The reversed-phase gradient LC/ESI-MS experiments were performed using an Agilent Zorbax Exlipse XDB-C8 column (2.1 mm×100 mm, particle size 1.8 μm), at a flow rate of 200 μl/min, and 25° C. A linear gradient using water/acetonitrile 95:5 (A) and water/acetonitrile (B), both with 0.05% acetic acid, following 1 min at 10% B and reaching 100% B in 25 min and held for 10 more minutes was employed.

### Statistical Analysis

Values are presented as means±SEM. Statistical differences between the treatments and controls were tested by unpaired two-tailed Student's t-test or one-way analysis of variance (ANOVA), followed by Bonferroni's posthoc testing, when appropriate. Analysis was performed using the GraphPad Prism 5.0 software. A difference of p<0.05 or less in the mean values was considered statistically significant.

### Example 1

### Effect of a Composition Comprising Sarcopoterium spinosum Extract on Pancreatic β-Cell Function

The RINm insulinoma cell line was used as a model for studying the effect of compositions comprising S. spinosum extract on β-cell function. The effects of S. spinosum compositions on pancreatic β-cell proliferation and insulin secretion were measured.

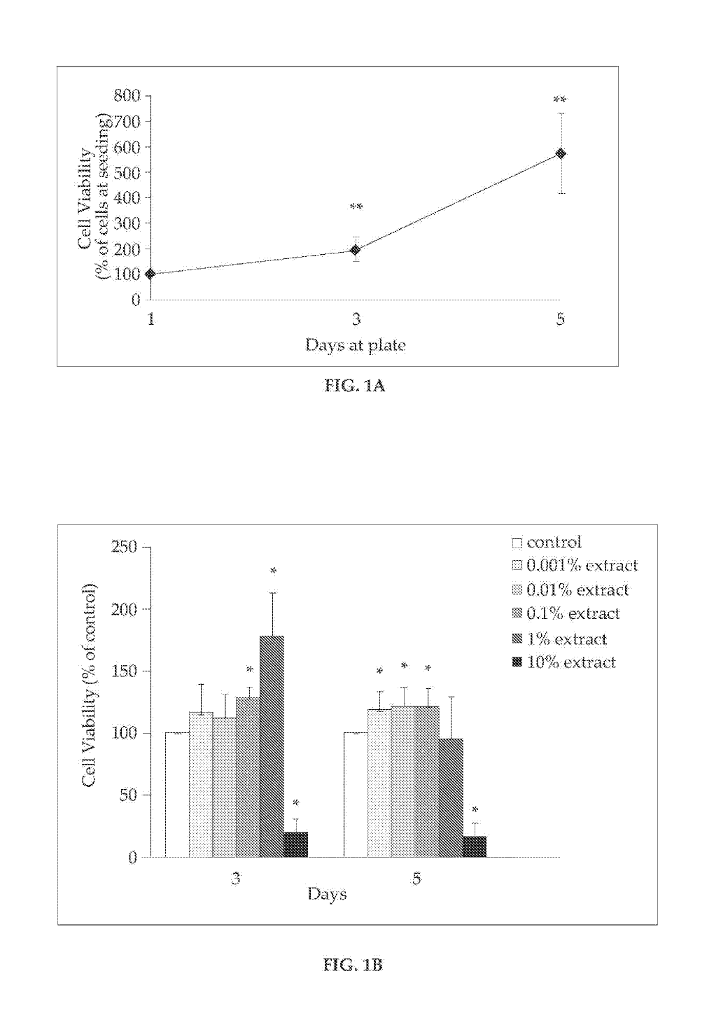

RINm cells were cultured on a 96-well plate, at a concentration of 5×10⁴ cells/ml in the absence or presence of compositions comprising different concentrations of S. spinosum extract (0.001-10% V/V). Proliferation was measured using a Cell Proliferation kit (XTT) 3 and 5 days after seeding, as described in the Materials and Methods section above. Results are presented in FIGS. 1A and 1B.

### Example 2

### Effect of Composition Comprising Sarcopoterium spinosum Extract on Insulin Secretion

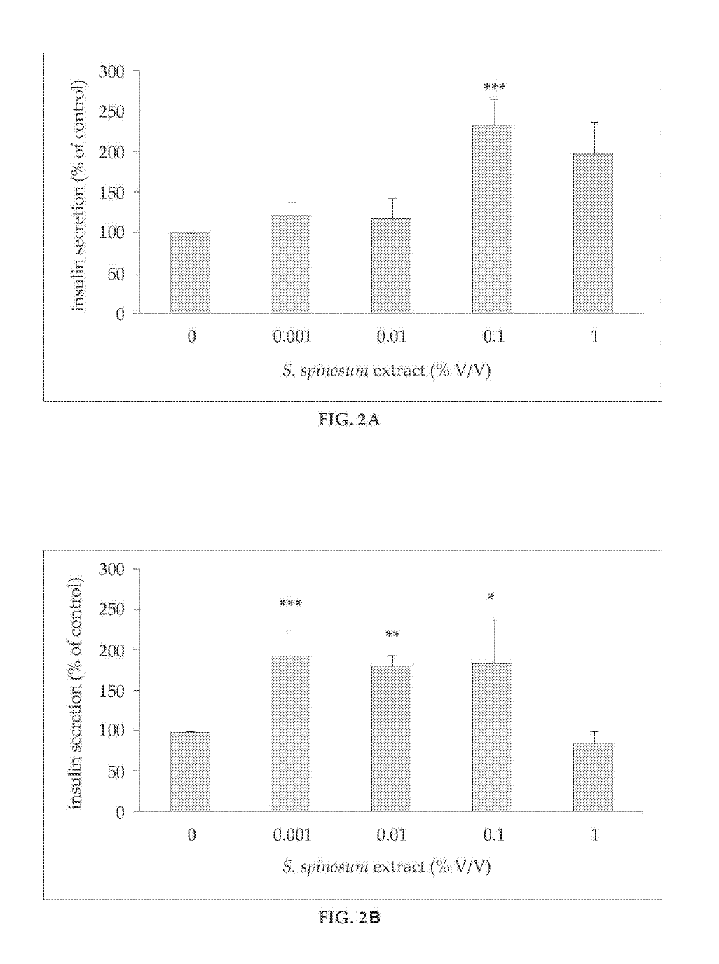

RINm pancreatic β-cells were treated with or without composition comprising Sarcopoterium spinosum extract (0.001-1% V/V) for 1 hour. Supernatant was collected at the basal state from unstimulated cells, and after glucose/forskolin induction of insulin secretion. The induction of insulin secretion was performed as described in the Materials and Methods section above. Insulin concentration was measured using the ELISA method. Results are presented in FIGS. 2A and 2B.

### Example 3

### Effect of Composition Comprising Sarcopoterium spinosum Extract on GSK3β Phosphorylation

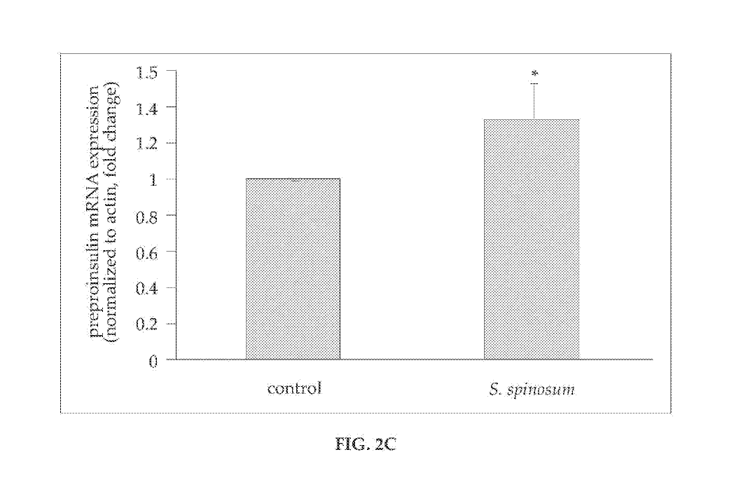

L6 myoblasts were induced to differentiate into myotubes. Differentiated myotubes were treated with a composition comprising 0.1 or 0.01% (V/V) S. spinosum extract for 20min, 1 h or 24 h. Cells were treated with 100 nM insulin for 20 min as a positive control. GSK-3β phosphorylation was measured using the ELISA detection kit. Western-blot analysis was performed using anti-pGSK (ser 9) or anti-actin. Optical density of the bands was performed using Scion-Image software. Results are presented in FIGS. 3A-B.

### Example 4

### Effect of Composition Comprising Sarcopoterium spinosum Extract on Lipolysis

Lipolysis was induced using the adrenergic agonist isoproterenol in order to analyze the extract's effect on lipolysis [20, 21], and the effect of compositions comprising S. spinosum extract on isoproterenol-induced lipolysis measured, with insulin as a positive control.

Differentiated adipocytes 3T3-L1) were treated with insulin (100 nM) or a composition comprising 1% (V/V) S. spinosum extract with or without the addition of isoproterenol (10 μM) for 60 min. Free fatty acid (FFA) concentration was measured by ANOVA.

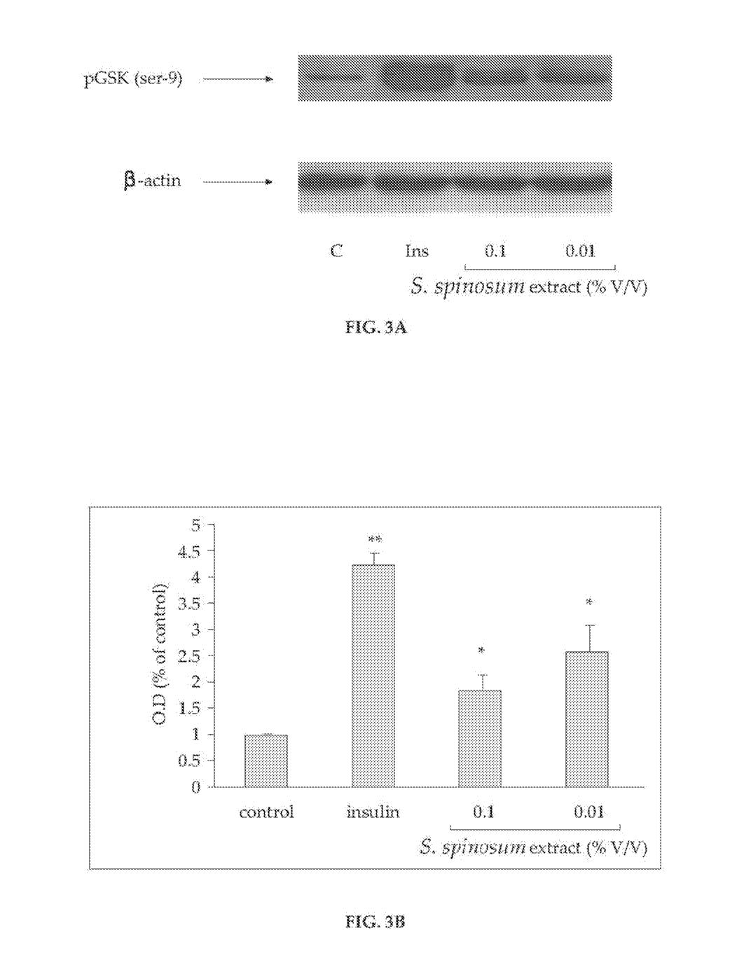

Results are presented in FIGS. 4A and 4B.

### Example 5

### Effect of Composition Comprising Sarcopoterium spinosum Extract on Glucose Uptake

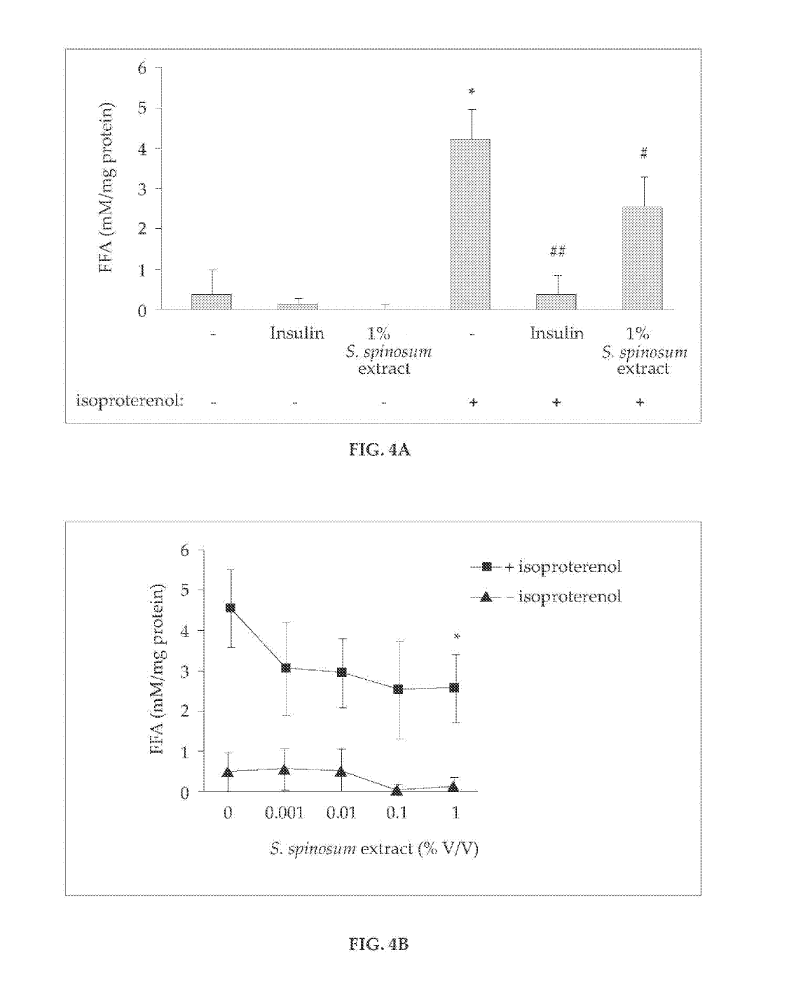

The effect of S. spinosum compositions on glucose uptake was measured in the AML12 hepatocyte cell line, L6 skeletal myoblasts and in differentiated 3T3-L1 adipocytes, and compared to the effect of insulin. Results are shown in FIGS. 5A-C.

Cells were transferred to serum-free, low glucose medium and stimulated with insulin as positive control or compositions comprising S. spinosum extract at concentrations of 0.001-1% (V/V) for 25 min. The uptake of [³H]2-deoxy-D-glucose into cells was determined as described in the Materials and Methods section.

### Example 6

### Effects of Compositions Comprising Sarcopoterium spinosum Extract on Glucose Levels In-Vivo, Using KK-A^{y} Mice

KK-A^{y} mice are a common model of type 2 diabetes mellitus (26, 29).

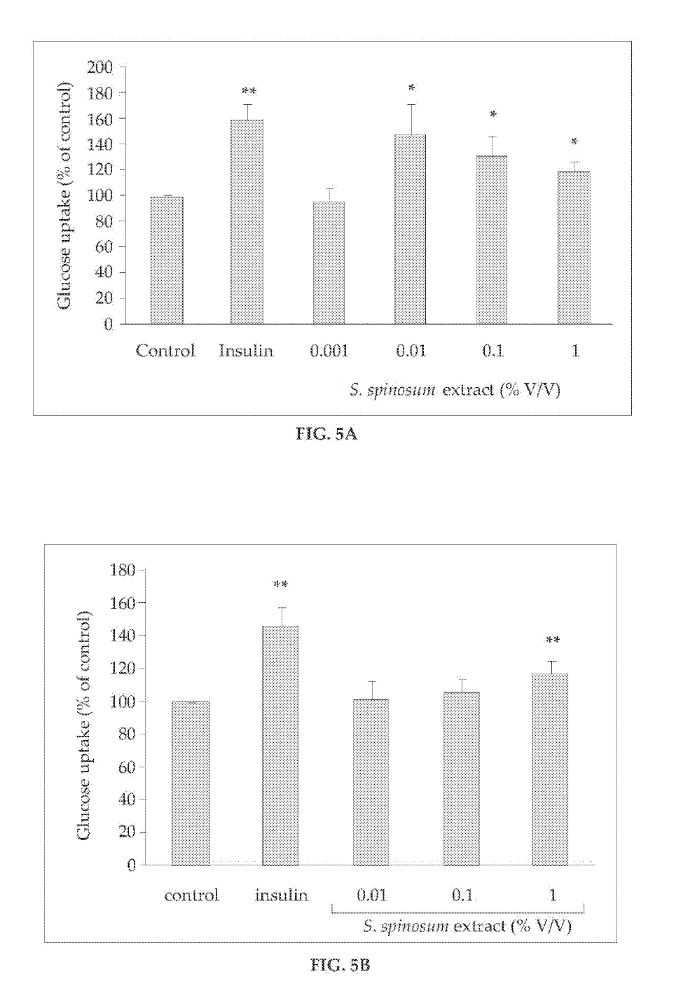

10 mice were given the composition comprising S. spinosum extract instead of drinking water, beginning at 6 weeks of age. Over the time of the experiment, the consumption of composition was found to be an average of 15 ml/mouse/day. At the age of 13 weeks and at an age of 18 weeks, intraperitoneal glucose tolerance tests (IPGTT) were performed. Fasting plasma glucose levels were measured as well as glucose levels at 15, 30, 60, 90 and 120 minutes following intraperitoneal glucose administration. The results for the 13 week-old mice are shown in FIG. 6. Similar results were obtained when IPGTT was measured at age of 18 weeks.

### Example 7

### Effects of Compositions Comprising Sarcopoterium spinosum Extract on Weight Gain and Food Intake In-Vivo

A first group of at least 10 normal mice was raised normally.

A second group of at least 10 normal mice were given the composition comprising S. spinosum extract instead of drinking water, beginning at 6 weeks of age.

A third group of at least 10 A^{y} mice was raised normally.

A fourth group of at least 10 A^{y} mice were given a composition comprising S. spinosum extract in water (100 gr/L, 10% dilution) instead of drinking water, beginning at 6 weeks of age.

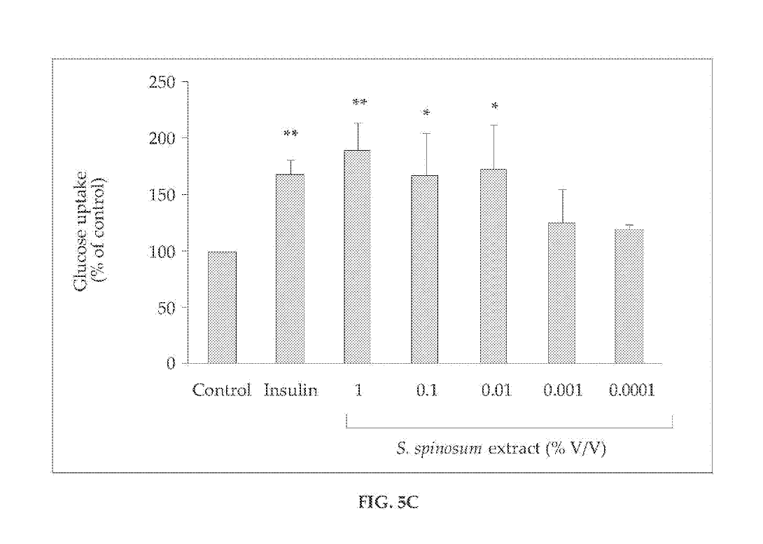

The mice of both groups were weighed at the age of 6 months. The average weight of the four groups are shown in FIG. 7 where the bar termed “yellow” represents the A^{y} mice groups (yellow coat) and the bar termed “black” represents the normal mice groups (black coat).

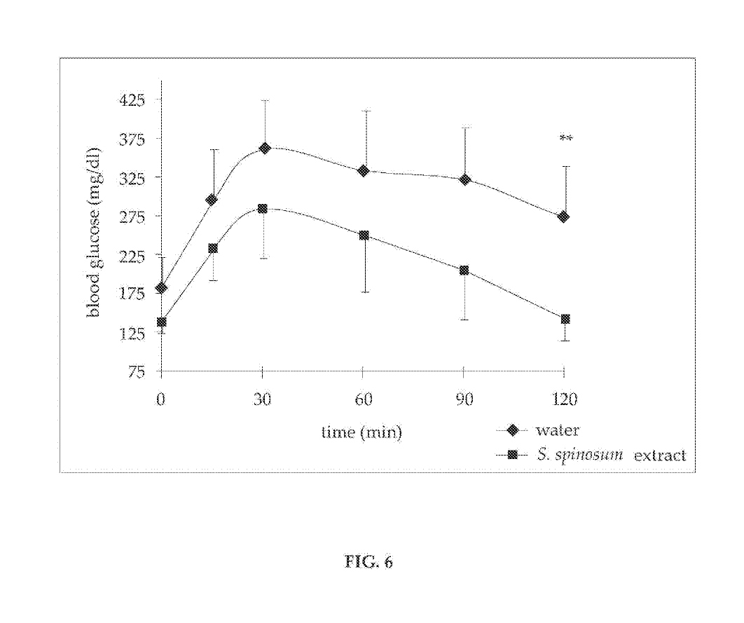

The average daily food intake of mice of both groups was monitored at the age of 6 months. The average daily food intake of the four groups are shown in FIG. 8 where the bar termed “yellow” represents the A^{y} mice groups (yellow coat) and the bar termed “black” represents the normal mice groups (black coat).

### Example 8

### Effects of Compositions Comprising Sarcopoterium spinosum Extract on Free Fatty Acid Levels In-Vivo

A first group of at least 10 A^{y} mice was raised normally.

A second group of at least 10 A^{y} mice were given the composition comprising S. spinosum extract instead of drinking water, beginning at 6 weeks of age.

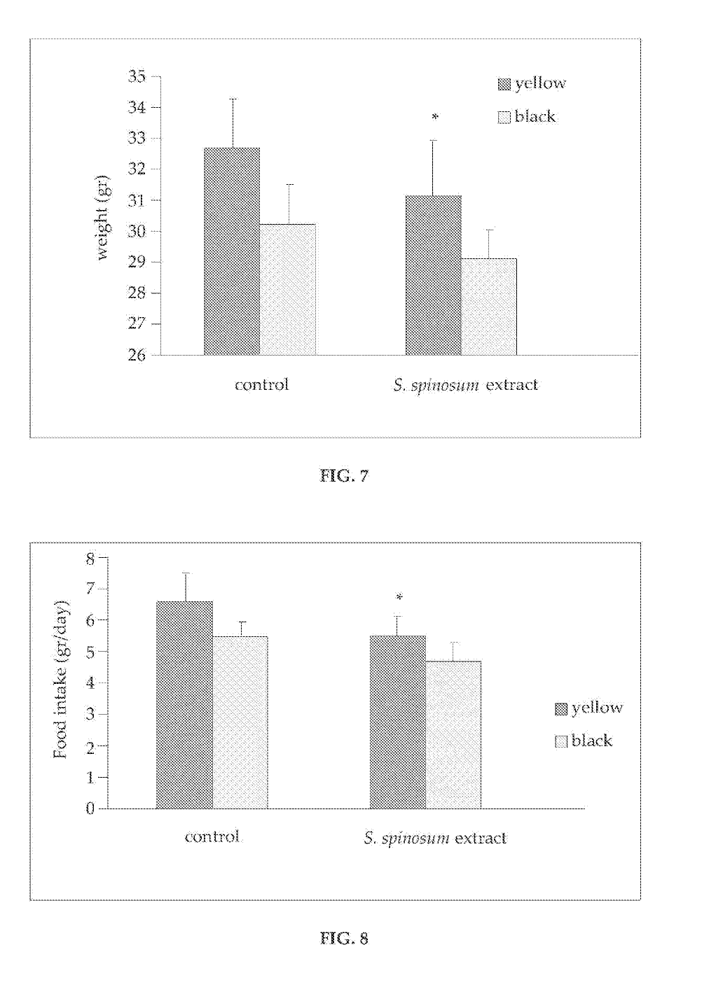

At the age of 19 weeks, fasting levels of free fatty acids in the blood of the mice of both groups was determined using a kit commercially available from Wako Chemicals USA, Inc. (Richmond Va., USA). The results are shown in FIG. 9.

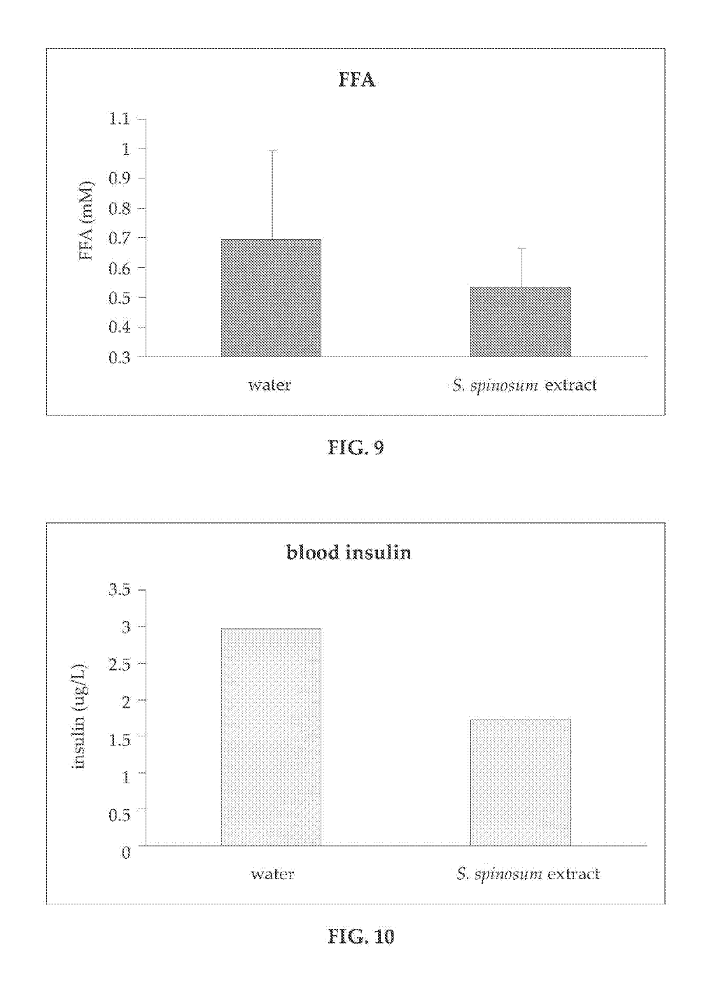

At the age of 19 weeks, fasting levels of insulin in the blood of the mice of both groups were determined using an ELISA kit commercially available from Mercodia (Uppsala, Sweden). The results are shown in FIG. 10.

### Example 9

### Effects of Composition Comprising Sarcopoterium spinosum Extract In-Vivo on Glucose Level in Diabetic KK-A^{y} Mice

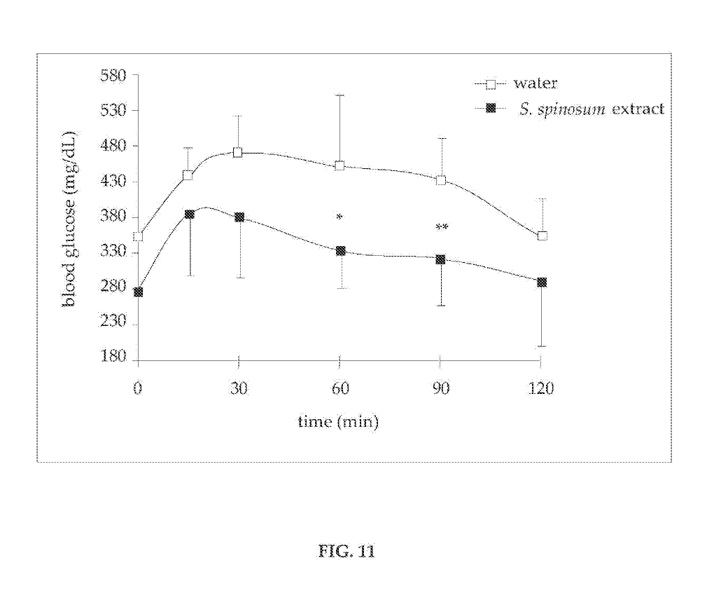

In order to evaluate the hypoglycemic effect of a composition comprising S. spinosum extract in mice that had already developed the disease, the effect of a 24 hour administration of the S. spinosum composition on blood glucose of diabetic male KK-A^{y} mice was examined. Glucose tolerance test was performed on mice at 13 weeks of age. Mice with a blood glucose level above 250 mg/dL at 120 min following glucose administration were considered to be diabetic. After 2 weeks, the diabetic mice were given the composition comprising S. spinosum extract instead of drinking water for 24 h before performing an additional glucose tolerance test. Mice were given an intraperitoneal injection of 1.5 mg glucose/g body weight after an 8-h fast. Blood glucose was determined at the indicated time from tail blood using the ACCU-CHEK Go Glucometer (Roche Diagnostics GmbH, Manheim, Germany). The results are shown in FIG. 11.

### Example 10

### Effect of Composition Comprising Sarcopoterium spinosum Extract In-Vivo on Development of Type 1 Diabetes

In order to evaluate the effect of S. spinosum on development of type I diabetes, NOD mice, which are susceptible to type 1 diabetes, were studied.

Two groups of mice were studied for a period of 30 weeks. The first (control) group were raised normally, including being allowed access to unlimited amounts of drinking water. The second (test) group were raised as for the first, but were given the composition comprising S. spinosum extract instead of drinking water,

After 6 weeks, the incidence of diabetes in the control group reached 20%, while the incidence of diabetes in the test group was only 5%.

After 28 weeks, the incidence of diabetes in the group group had stabilized at 70% of the individuals. Significantly fewer animals in the test group developed type 1 diabetes.

### Example 11

### Identification of Active Components of Sarcopoterium spinosum Extract

Bioactivity guided fractionation of hot water extract is performed using stepwise solid-phase extraction and reversed phase chromatography methods, both preparative and analytical. Fractions are eluted using graded concentrations of ethanol in water, the solvent is vacuum-evaporated, and residues are re-dissolved in the appropriate solvent. Optionally, the solvent volume only is reduced to avoid recovery problems. The active fraction, alone or in combination with other fraction is separated further for the identification of the specific active compounds.

Once a close to pure fraction is isolated, mass spectrometry is used for the primary identification of the included compounds.

Each fraction is assayed for its activity using the biological tests used for the measurements of the activity of the whole extract, as described above, including measurement of insulin secretion and proliferation of β-cells, glucose uptake by L6 , AML12 and 3T3-L1 and lipolytic activity of 3T3-L1. Thus, the active fraction, (alone or in combination with other fractions) mediating each biological function is identified. The active fractions are analyzed further for identification of the specific active chemical compounds.

The isolated active compounds are measured for their beneficial activity in-vivo, using mice models of diabetes (A^{Y} and NOD mice).

The molecular mechanism of action of the active compounds are elucidated by treating cellular in-vitro models of β-cells, skeletal muscle and adipocytes with the active compounds. Changes in gene as well as protein expression profile are followed using PCR and protein array strategies. Specific genes and protein that are found to be regulated by active compounds from the herb extract, are analyzed again using real time PCR and western blot analysis, respectively.

### Example 12

### Increase of Fertility of Diabetic Females

Two groups of diabetic yellow female mice are kept in contact with healthy black male mice. The first group is provided with drinking water while the second group is provided with a composition comprising an S. spinosum extract instead of drinking water.

Few, if any, pregnancies are observed in the first group.

In the second group, the rate of pregnancies is similar to that found in a group of healthy black females kept in contact with healthy black male mice.

After the drinking water of the first group is replaced with the S. spinosum composition, the rate of pregnancies of the first group substantially equals that of the second group.

### RESULTS

Effect of Sarcopoterium spinosum Composition on Pancreatic β-cell Function

Cell proliferation studies on the RINm insulinoma cell line using the XTT method revealed an increase of 197±44.4 and 578±112 percent in OD on the third and fifth day, respectively, compared to the day of seeding, indicating an increase in cell proliferation (FIG. 1A).

As can be seen in FIG. 1B, the S. spinosum compositions of 0.1 and 1% (V/V) increased cell proliferation by 129±8.37 and 178±35 percent, respectively, compared to control untreated cells. On the fifth day after seeding, proliferation increased in cells treated with more dilute compositions (0.001, 0.01 and 0.1% V/V; 118±16.21, 122±15.14 and 123±13.18 percent increase in proliferation compared to control, respectively) but not in cells treated with 1% V/V composition. The lack of response to 1% V/V composition on the fifth day compared to the third day may result from contact inhibition that abrogates the effect of the extract. A more concentrated composition (10%) induced cell death. The lethal concentration was not used in further experiments. Results are mean±SEM of 3 independent experiments. *p<0.05, **p<0.005, compared to untreated cells.

Insulin secretion in the basal state (2.8 mM glucose), was increased by 232±23.8 percent in cells treated with a composition comprising 0.1% (V/V) of the S. spinosum extract compared to the untreated cells (FIG. 2A). Higher and lower doses were less effective. An increased glucose concentration (15 mM), given with 10 μM forskolin, induced insulin release. However, the glucose/forskolin induced insulin secretion was even higher in cells treated with 0.001%, 0.01% and 0.1% (V/V) S. spinosum extract (193±30.1, 180±14.2 and 184±55.6 percent, respectively) compared to control cells (FIG. 2B). The mean absolute concentration of insulin measured was 0.97 μg/L in the basal state, and 1.97 μg/L following glucose/forskolin induction. The compositions did not affect insulin secretion when given 24 h before measurement of insulin secretion (data not shown). Results are mean±SEM of 3 independent experiments. *p<0.05, **p<0.005, ***p<0.0005 compared to untreated cells.

S. spinosum compositions comprising a 0.1 mg/ml concentration of S. spinosum extract increased preproinsulin mRNA expression by 1.32±0.203-fold compared to control (FIG. 2C).

Effect of Sarcopoterium spinosum Compositions on GSK3β Phosphorylation

As shown in FIG. 3A, insulin induced a 4-fold increase in GSK3β ser-9 phosphorylation compared to the control. The effect of the S. spinosum compositions is similar to that of insulin, but at a lower magnitude (around 2-fold greater than the control). Only an acute treatment (20 min or 1 h) with the compositions was effective. Myotubes treated with the extract for 24 h did not demonstrate any increase in GSK3β phosphorylation. Similar results were obtained by measuring phospho-GSK3β using the ELISA phosphodetection kit, as shown in FIG. 3B.

Effect of Sarcopoterium spinosum Compositions on Lipolysis.

FIG. 4A shows that free fatty acid release is very low in unstimulated adipocytes, and is not affected by insulin or S. spinosum composition. Administration of isoproterenol for one hour increased lipolysis and induced the release of 4.23±3.01 mM FFA/mg protein, *p<0.005, compared to control, untreated cells. #p<0.05, ##p<0.005 compared to isoproterenol treated cells (FIG. 4A). As expected, insulin blocked the isoproterenol-induced lipolysis (0.47±0.16 mM FFA/mg protein) (11). The S. spinosum composition (1% V/V) had an effect similar to that of insulin, and resulted in a decrease in free fatty acid release to 2.5±2.32 mM FFA/mg protein (FIG. 4A).

FIG. 4B shows the dose response curve (0.001-1% V/V) of S. spinosum composition on isoproterenol-induced free fatty acid release. Results are mean±SEM of 5 independent experiments. *p<0.05 compared to isoproterenol treated cells, without S. spinosum composition, by ANOVA Lower concentrations were less effective (FIG. 4B).

### Effect of Sarcopoterium spinosum Composition on Glucose Uptake

As shown in FIG. 5A, the S. spinosum compositions (0.01, 0.1 and 1% V/V) exhibited an insulin-like effect on glucose uptake in hepatocytes by inducing a 148±10, 133±23 and 119±14 percent increase in glucose uptake, respectively, compared to 160±12 percent increase in glucose uptake obtained by insulin. Each bar represents the mean±SEM of a measurement made on three replicates in each of 5 experiments. *p<0.05, **p<0.0005 compared to control, untreated cells. Data are expressed as percent of basal uptake in control cells.

A composition comprising 0.01% (V/V) S. spinosum extract was more effective than lower or higher doses in these cells. Similar results were found in L6 myoblasts and in differentiated 3T3-L1 adipocytes. In L6 myoblasts, the S. spinosum composition (1% V/V) induced a slight but significant increase in glucose uptake of 119±7.27 percent, compared to a 146±11.26 percent increase induced by insulin (FIG. 5B). In 3T3-L1 adipocytes, the S. spinosum compositions (0.01, 0.1 and 1% V/V) induced a 172±40, 165±39 and 189+24 percent increase in glucose uptake, respectively, compared to 167±13 percent increase in glucose uptake obtained by insulin (FIG. 5C).

Effects of a Composition Comprising Sarcopoterium spinosum Extract In-Vivo

Plasma glucose levels at 120 min following IPGTT, were significantly lower (p<0.05) in mice receiving the extract compared to untreated mice (141.6±26 and 272.7±67 respectively). There was no significant reduction in fasting glucose concentrations (FIG. 6).

Mice, whether normal or susceptible to developing type 2 diabetes, administered a composition comprising Sarcopoterium spinosum extract had a lower average weight than the control group, FIG. 7. It can be concluded that in some embodiments, administration of composition comprising Sarcopoterium spinosum extract has a beneficial effect, for example relating to obesity, weight gain and related conditions.

Mice, whether normal or susceptible to developing type 2 diabetes, administered a composition comprising Sarcopoterium spinosum extract had a lower average food intake than the control group, FIG. 8. It can be concluded that in some embodiments, administration of composition comprising Sarcopoterium spinosum extract has a beneficial effect, for example relating to obesity, weight gain, overeating and related conditions and may lead to increased longevity.

Mice administered a composition comprising Sarcopoterium spinosum extract had lower average fasting free fatty acid concentrations in the blood than the control group, FIG. 9. It can be concluded that in some embodiments, administration of composition comprising Sarcopoterium spinosum extract has a beneficial effect, for example relating to general health, blood profile and athersclerosis.

Mice administered a composition comprising Sarcopoterium spinosum extract had lower average fasting insulin concentrations in the blood than the control group, FIG. 10. It can be concluded that in some embodiments, administration of composition comprising Sarcopoterium spinosum extract has a beneficial effect, for example relating to blood profile and diabetes.

Effects of Composition Comprising Sarcopoterium spinosum Extract In-Vivo on Glucose Level in Diabetic KK-A^{y} Mice

In order to evaluate the hypoglycemic effect of a composition comprising S. spinosum extract in mice that had already developed the disease, the effect of a 24 hour administration of the S. spinosum composition on blood glucose of diabetic male KK-Ay mice was examined and the results shown in FIG. 11. As noted above, IPGTT was performed on 13 weeks old mice and two weeks later, the diabetic mice were given S. spinosum composition for 24 h before performing a second IPGTT. In FIG. 11, data are expressed as the mean±SEM of 8-10 animals. *p<0.05 and **p<0.005, as compared with control group by two-way ANOVA.

As can be seen in FIG. 11, the short-term administration of S. spinosum composition did not reduce fasting glucose level, but led to improved glucose tolerance compared to vehicle-treated mice.

Analysis of Bioactive Compounds in Sarcopoterium spinosum Extract

The active compounds catechin and epicatechin were identified in the bioactive extract, by retention times, accurate mass (measured mass 289.07181, theoretical 289.07176) and MS/MS spectrum. Compounds with m/z 577.13531 were also isolated in the sample, with retention times of the three most intense peaks being 5.6, 6,8, and 8.4 min. The accurate mass m/z 5.7713525 is consistent with an elemental composition C3OH25012, suggesting a dimer form of catechin.

From the results it is seen that in some embodiments, administration of a composition comprising S. spinosum has insulin-like effects in skeletal muscle, adipose tissue and hepatocytes, which are classic target tissues of insulin and play important roles in the maintenance of glucose homeostasis. The extract increases glucose uptake in hepatocytes, adipocytes and myotubes. The extract also increases GSK3β phosphorylation in myotubes, an event known to be crucial for glycogen synthesis which is a major anabolic pathway activated by insulin. Furthermore, the extract inhibited isoproterenol-induced lypolysis in adipocytes, as occurs in response to insulin. The extract increased basal as well as glucose/forskolin-induced insulin secretion.

From the results it is seen that in some embodiments, administration of a composition comprising S. spinosum extract has a number of beneficial effects including weight control and reduced food intake that in some embodiments has beneficial effects such as treating and preventing obesity and overweight, increasing general health, and increasing longevity.

From the results it is also seen that in some embodiments, administration of a composition comprising S. spinosum extract has a number of beneficial effects including lower free fatty acid and insulin blood concentration that in some embodiments, has beneficial effects such as improving blood chemistry, preventing (or delaying the onset of) atherosclerosis and preventing (or delaying the onset of) type 2 diabetes in subjects, especially subjects susceptible thereto.

As discussed above, in some embodiments an S. spinosum composition is prepared by diluting (0.001% to 10%) a water extract of S. spinosum prepared according to the methods known in the art. In some embodiments, an S. spinosum extract composition is prepared by reconstituting (1 g of dry extract in 0.1-10000 liter compostion) a dried (e.g., by lyophilization) water extract of S. spinosum prepared according to the methods known in the art. Surprisingly, in some embodiments the dilute compositions are more effective or are less toxic than the known concentrated extract when used as a composition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

### REFERENCES

1. Cicero A F, Derosa G, Gaddi A “The hypoglycaemic and antihyperglycaemic effect of citrullus colocynthis fruit aqueous extract in normal and alloxan diabetic rabbits” Acta Diabetol 2004, 41, 91-98.
2. Saad B, Azaizeh H, Said O “Tradition and perspectives of Arab herbal medicine: A review” Evidence-Based Complementary and Alternative Medicine 2005, 2, 475-479.
3. Friedman J, Yaniv Z, Dafni A, Palewitch D “A Preliminary Classification of the Healing Potential of Medicinal-Plants, Based on A Rational Analysis of An Ethnopharmacological Field Survey Among Bedouins in the Negev Desert, Israel” J Ethnopharmacol 1986, 16, 275-287.
4. Ivorra M D, Paya M, Villar A “A review of natural products and plants as potential antidiabetic drug” J Ethnopharmacol 1989, 27, 243-275.
5. Dafni A, Yaniv Z, Palevitch D “Ethnobotanical Survey of Medicinal-Plants in Northern Israel” J Ethnopharmacol 1984, 10, 295-310.
6. Hamdan I I, Afifi F U “Studies on the in vitro and in vivo hypoglycemic activities of some medicinal plants used in treatment of diabetes in Jordanian traditional medicine” J Ethnopharmacol 2004, 93, 117-121.
7. Ali-Shtayeh M S, Yaniv Z, Mahajina J “Ethnobotanical survey in the Palestinian area: a classification of the healing potential of medicinal plants” 2000 J Ethnopharmacol 123, 45-50.
8. Said O, Khalil K, Fulder S, Azaizeh H “Ethnopharmacological survey of medicinal herbs in Israel, the Golan Heights and the West Bank region” J Ethnopharmacol 2002, 83, 251-265.
9. Yaniv Z, Dafni A, Friedman J, Palevitch D “Plants Used for the Treatment of Diabetes in Israel” J Ethnopharmacol 1987, 19, 145-151.
10. Henkin Z, Seligman N G “Survival of Sarcopoterium Spinosum seedlings growing on terra rossa soil” Israel Journal of Plant Sciences 2007, 55, 45-51.
11. Shani J, Joseph B, Sulman F G “Fluctuations in the hypoglycaemic effect of Poterium spinosum L. (Rosaceae)” Arch Int Pharmacodyn Ther 1970, 185, 344-349.
12. Mishkinsky J S, Goldschmied A, Joseph B, Ahronson Z, Sulman F G “Hypoglycaemic effect of Trigonella foenum graecum and Lupinus termis (leguminosae) seeds and their major alkaloids in alloxan-diabetic and normal rats” Arch Int Pharmacodyn Ther 1974, 210, 27-37.
13. Kanter Y, Teicher D, Wiener F, Lasman T, Mandelbaum A “The Response to Oral Glucose-Load Among Diabetic and Normal Rats, Primed with Poterium-Spinosum Extracts” Israel Journal of Medical Sciences 1983, 19, 304.
14. Reher G, Reznicek G, Baumann A “Triterpenoids from Sarcopoterium spinosum and Sanguisorba minor” Planta Med 1991, 57, 506.
15. Reher G, Budesinsky M “Triterpenoids from Plants of the Sanguisorbeae 1” Phytochemistry 1992, 31, 3909-3914.
16. Frost S C, Lane M D “Evidence for the involvement of vicinal sulfhydryl groups in insulin- activated hexose transport by 3T3-L1 adipocytes” J Biol Chem 1985, 260, 2646-2652.
17. Frame S, Zheleva D “Targeting glycogen synthase kinase-3 in insulin signalling” Expert Opin Ther Targets 2006, 10, 429-444.
18. Henriksen E J, Dokken B B “Role of glycogen synthase kinase-3 in insulin resistance and type 2 diabetes” Curr Drug Targets 2006, 7, 1435-1441.
19. Large V, Peroni O, Letexier D, Ray H, Beylot M “Metabolism of lipids in human white adipocyte” Diabetes Metab 2004, 30, 294-309.
20. Yin W, Mu J, Birnbaum M J “Role of AMP-activated protein kinase in cyclic AMP-dependent lipolysis In 3T3-L1 adipocytes” J Biol Chem 2003, 278, 43074-43080.
21. Kralisch S, Klein J, Lossner U, Bluher M, Paschke R, Stumvoll M et al. “Isoproterenol, TNFalpha, and insulin downregulate adipose triglyceride lipase in 3T3-L1 adipocytes” Mol Cell Endocrinol 2005, 240, 43-49.
22. Bouche C, Serdy S, Kahn C R, Goldfine A B “The cellular fate of glucose and its relevance in type 2 diabetes” Endocr Rev 2004, 25, 807-830.
23. Houghton P J, Howes M J, Lee C C, Steventon G “Uses and abuses of in vitro tests in ethnopharmacology: visualizing an elephant” J Ethnopharmacol 2007, 110, 391-400.
24. Maedler K, Donath M Y “Beta-cells in type 2 diabetes: a loss of function and mass” Horm Res 2004, 62 Suppl 3, 67-73.
25. Ahren B “Type 2 diabetes, insulin secretion and beta-cell mass” Curr Mol Med 2005, 5, 275-286.
26. Chen Z H. et al. “Saponins isolated from the root of Panax notoginseng showed significant anti-diabetic effects in KK-Ay mice” Am J Chin Med 2008, 36(5),939-51.
27. Kwon, D. Y., et al. “Long-term consumption of saponins derived from Platycodi radix (22 years old) enhances hepatic insulin sensitivity and glucose-stimulated insulin secretion in 90% pancreatectomized diabetic rats fed a high-fat diet” British Journal of Nutrition 2009, 101(3), 358-366.
28. Jiang, B. R., et al. “Astragaloside IV Attenuates Lipolysis and Improves Insulin Resistance Induced by TNF alpha in 3T3-L1 Adipocytes” Phytotherapy Research 2008, 22(11),1434-1439.
29. Im R., et al. “Mechanisms of blood glucose-lowering effect of aqueous extract from stems of Kothala himbutu (Salacia reticulata) in the mouse” J Ethnopharmacol. 2009, 121(2), 234-40.

1-8. (canceled)

9. A method of preventing or delaying the onset of diabetes in a subject, the method comprising administering a pharmaceutically-effective amount of an extract of S. spinosum to said subject.

10. The method of claim 9, wherein said diabetes is selected from the group consisting of: type 1 diabetes: and type 2 diabetes.

11-29. (canceled)

30. The method of claim 9, wherein the subject is selected from the group consisting of: non-diabetics; and subjects susceptible to the development of diabetes.

31-32. (canceled)

33. The method of claim 9, wherein said extract of S. spinosum is selected from the group consisting of: complete extracts from parts of S. spinosum; a pharmaceutically-active component of such extracts; and a combination of two or more such pharmaceutically-active components.

34. The method of claim 33, wherein at least one of said pharmaceutically-active components is isolated from a natural extract of S. spinosum

35. The method claim 33, wherein at least one of said pharmaceutically-active components is selected from the group consisting of: catechin; and epicatechin.

36. The method of claim 9, wherein said extract is a hot water extract.

37. The method of claim 9, wherein said extract comprises an extract of a root of S. spinosum.

38. A method of achieving an effect on a physiological function in a subject in need thereof, the method comprising: administering a pharmaceutically-effective amount of an extract of S. spinosum to said subject, wherein said effect is selected from the group consisting of: increasing pancreatic cell proliferation; inducing glucose uptake in a cell; increasing fertility of a diabetic female; improving glucose tolerance; preventing or delaying the onset of atherosclerosis; decreasing weight gain; decreasing food consumption; increasing longevity; increasing libido in a female subject; treating erectile dysfunction in a male subject; preventing or reducing obesity.

39. The method of claim 38 for inducing glucose uptake in a cell, wherein said cell is selected from the group consisting of: an adipocyte; a hepatocyte; and a myotube.

40. The method of claim 38, wherein said subject is susceptible to development of diabetes.

41. The method of claim 38, wherein said subject is diabetic.

42. The method of claim 38, wherein said subject is not diabetic.

43. The method of claim 38, wherein said subject is susceptible to development of hyperlipidemia.

44. The method of claim 38, wherein said subject has hyperlipidemia.

45. The method of claim 38, wherein said extract of S. spinosum is selected from the group consisting of: complete extracts from parts of S. spinosum; pharmaceutically-active components of such extracts; and combinations of two or more such pharmaceutically-active components.

46. The method of claim 38, wherein at least one of said pharmaceutically-active components is isolated from a natural extract of S. spinosum.

47. The method of claim 38, wherein at least one said of pharmaceutically-active components is selected from the group consisting of: catechin; and epicatechin.

48. The method of claim 38, wherein said extract is a hot water extract.

49. The method of claim 38, wherein said extract comprises an extract of a root of S. spinostum.
